(19) United States
(12) Patent Application Publication
(10) Pub. No.: US 2026/0182994 A1
(43) Pub. Date: Jul. 2, 2026

(54) DUAL-STAGE CROSSLINKING ENDOVASCULAR EMBOLIC AND METHOD OF USE

(71) Applicant: STRYKER CORPORATION, Portage, MI (US)

(72) Inventors: Karen Dubbin, Portage, MI (US); Andrew Lankenau, Portage, MI (US); Stephen Porter, Piedmont, CA (US)

(73) Assignee: STRYKER CORPORATION, Portage, MI (US)

(21) Appl. No.: 19/547,530

(22) Filed: Feb. 23, 2026

Related U.S. Application Data
(63) Continuation of application No. PCT/US2025/026815, filed on Apr. 29, 2025.
(60) Provisional application No. 63/642,462, filed on May 3, 2024.

(51) Int. Cl. A61B 17/12; A61L 24/00; A61L 24/02; A61L 24/04

(52) U.S. Cl. CPC A61B 17/12186; A61B 17/12113; A61L 24/0031; A61L 24/02; A61L 24/043; A61B 2017/1205; A61L 2400/06

(57) ABSTRACT

An embolic hydrogel for use with a patient comprise an aqueous buffer and at least one functionalized polymer respectively having at least one polymer backbone chemically modified with one or more functional groups. The functional group(s) facilitate formation of primary reversible crosslinks on the polymer backbone(s), such that the embolic hydrogel transitions from a gel state to a viscous liquid state in response to a shear force applied to the embolic hydrogel, and transitions from the viscous liquid state back to the gel state in response to an absence of a shear force applied to the embolic hydrogel. The embolic hydrogel further comprises at least one phase-transitioning polymer that creates secondary crosslinks therein in response to a physiological environment, such that the embolic hydrogel transitions from the gel state to a solid state.

### RELATED APPLICATION DATA

The application is a continuation of International Patent Application No. PCT/US2025/026815, filed on Apr. 29, 2025, which claims the benefit of U.S. Provisional Patent Application Ser. No. 63/642,462, filed on May 3, 2024, the entire disclosures of all of which are hereby incorporated herein by reference in their entirety into the present application.

### FIELD

The present disclosure relates generally to endovascular embolization, and more particularly, to embolic hydrogels for injection into a patient for treating vascular defects.

### BACKGROUND

In many clinical situations, blood vessels are occluded for a variety of purposes, such as to control bleeding in the case of hemorrhage or hematoma, prevent the flow of blood to a tumor (i.e., tumor devascularization), or to treat a diseased blood vessel, such as an arteriovenous malformation (AVM), an arteriovenous fistula, or an aneurysm. Transcatheter arterial embolization is a widely used approach for the treatment of such hemorrhages or hematomas, tumors, and vascular diseases.

Transcatheter arterial embolization involves initially positioning a small profile delivery catheter or micro-catheter at a vascular target site using a guidewire. It is desirable that the lumen, and thus the outer diameter, of the delivery catheter be as small as possible to allow the vascular target site to be accessed through a very small vasculature. The guidewire may then be removed from the delivery catheter, and embolic material introduced through the lumen of the delivery catheter at the vascular target site.

In one transcatheter arterial embolization procedure, solid embolic material in the form of expandable metallic coils are delivered through the delivery catheter at the vascular target site. The coils then expand, thereby filling and occluding the vascular target site. Coils are considered to be effective in controlled and precise deployment at the vascular target site. However, the delivery of coils at a vascular target site is time consuming, because it often requires fluoroscopy after placement of coil to ensure that the coil is properly located. In addition, the proper size for the coils normally needs to be determined and selected prior to implantation. Furthermore, it may be difficult to achieve complete occlusion at the vascular target site using coils, which may compact over time, leaving cavities for subsequent revascularization. Also, these metallic coils can produce extensive streak artifacts, and thus, cause interference of accurate follow-up assessment of the treated area with imaging techniques, including fluoroscopy, computerized tomography (CT), and magnetic resonance imaging (MRI).

In another transcatheter arterial embolization procedure, liquid embolic material is delivered through the delivery catheter at the vascular target site, which then solidifies in and occludes the vascular target site. In one known technique, the liquid embolic material may include two liquid components that are delivered from separate sources at the vascular target site. For example, the two liquid components can be delivered through two independent lumens of a catheter. Upon contact with each other, the liquid components react and solidify into an embolic mass, thereby occluding the vascular target site. In contrast to solid embolic material, liquid embolic material induces quick occlusion at the vascular target site. Furthermore, liquid embolic material may easily carry pharmacological agents and/or radioisotopes to provide additional therapy. However, also in contrast to solid embolic material, liquid embolic material is difficult to deploy in a controlled manner, and may incidentally embolize non-targeted vessels and/or cause entrapment of the delivery catheter, which may lead to surgical difficulties. As result, complications of recanalization and incomplete embolization can occur. Furthermore, existing liquid embolic delivery systems that require delivery of more than one liquid component to the distal portion of the delivery catheter may be cumbersome and may have limited utility due to the need to have more than one independent lumen in the delivery catheter. In addition, liquid embolic delivery systems may be expensive to develop and manufacture due to the need to fabricate multiple components that are specifically required to address the dynamics and kinetics involved with consistently mixing the liquid components in such delivery systems. Furthermore, when more than one liquid component is delivered at the vascular target site, there is a risk that the liquid components may not be completely combined at the vascular target site to form the desired embolic composition. As a result, the unmixed portion of either one of the liquid components may dissipate into the blood stream or travel to other locations within the body. Furthermore, inconsistent mixing of the liquid components will result in high deployment-to-deployment variations.

In still another transcatheter arterial embolization procedure, the liquid embolic is delivered as one liquid component containing a polymer suspended in a solvent. As the solvent is carried away from the polymer component via diffusion and blood flow, the polymer component solidifies leaving a solid cast of the vessel. This mechanism relies on the presence of solvents, some of which can have toxic or painful effects on the patient. Additionally, the solidification of the embolic is controlled by diffusion, limiting the user's control over delivery timing and distal penetration.

In yet another transcatheter arterial embolization procedure, the liquid embolic contains a monomer that is mixed with a contrast agent immediately before delivery. Once in the blood stream, the monomer polymerizes into an adhesive polymer. In this technique, the user faces risk of the catheter becoming entrapped in the adhesive polymer during delivery.

To address the aforementioned issues of solid and liquid embolic materials, injectable embolic hydrogels that can be introduced at vascular target sites via a delivery catheter in a more controlled manner without toxic or painful solvents or risk of catheter entrapment have been developed. To allow injectable embolic hydrogels to be delivered through a small diameter delivery catheter, such as those used for delivering embolic material at intracranial vascular target site, such embolic hydrogels may have a crosslinking mechanism that allows for the embolic hydrogel to be shear-thinning in response to being injected through the delivery catheter. Some embolic hydrogels, despite being considered injectable, either do not have a sufficiently low enough viscosity for injection through a relatively small diameter delivery catheter intended to be delivered into the cranial vasculature of a patient, or do not solidify enough to effectively occlude the vascular target site.

In some cases, an initiator can be added to a relatively low-viscosity embolic hydrogel, such that, after being delivered through a relatively small diameter delivery catheter at the vascular target site, the embolic hydrogel will sufficiently solidify to effectively occlude the vascular target site. In some cases, a time-based initiator may be added to the embolic hydrogel prior to injection through the delivery catheter, such that the embolic hydrogel solidifies after a certain time has elapsed. However, if the embolic hydrogel with the additive initiator is not injected fast enough at the vascular target site, the embolic hydrogel may prematurely solidify, thereby making injection of the embolic hydrogel through the delivery catheter impossible. In other cases, the initiator may be added to the embolic hydrogel after delivery at the vascular target site, such that the embolic hydrogel timely solidifies only at the vascular target site. However, the addition of the initiator would require an additional lumen in the delivery catheter or the addition of another catheter, thereby requiring a specialized catheter and/or further complicating the embolic delivery procedure.

There, thus, remains an ongoing need for an improved means for endovascularly delivering an embolic hydrogel into a patient.

### SUMMARY

In accordance with one aspect of the present inventions, an embolic hydrogel for use with a patient is provided.

The embolic hydrogel comprises an aqueous buffer, and at least one functionalized polymer respectively having at least one polymer backbone chemically modified with one or more functional groups. Each of the polymer backbones may comprise, e.g., a linear, branched, or multi-arm structure. The functional groups(s) facilitate formation of primary reversible crosslinks (e.g., covalent) on the polymer backbone(s), such that the embolic hydrogel transitions from a gel state to a viscous liquid state in response to a shear force applied to the embolic hydrogel, and transitions back from the viscous liquid state to the gel state in response to an absence of a shear force applied to the embolic hydrogel. In one example, the embolic hydrogel transitions from the viscous liquid state to the gel state within fifteen seconds in response to the absence of the shear forces after injection into the patient.

In one embodiment, the functional group(s) comprises at least two different functional groups that interact act with each other to form the primary reversible crosslinks on the polymer backbone(s). In one example of this embodiment, the functionalized polymer(s) may comprise a first functionalized polymer and a second functionalized polymer. In this case, the polymer backbone of the first functionalized polymer may be chemically modified with a first one of the different functional groups, and the second functionalized polymer may be chemically modified with a second one of the different functional groups. The different functional groups may interact with each other to form the primary reversible crosslinks between the respective polymer backbones of the first functionalized polymer and the second functionalized polymer. In another example of this embodiment, the functionalized polymer(s) may comprise a single functionalized polymer. In this case, the polymer backbone of the single functionalized polymer may be chemically modified by the different functional groups that interact with each other to form the primary reversible crosslinks within the polymer backbone of the single functionalized polymer. In any of these examples, a first one of the different functional groups may be ketones, aldehydes, benzaldehydes, or derivatives thereof, while a second one of the different functional groups may be amine, hydrazide, acylhydrazide, aminooxy, or derivatives thereof.

In another embodiment, the functionalized polymer(s) may comprise a single functionalized polymer, and the functional group(s) may comprises a metal coordinating ligand (e.g., thiol, iminodiacetate, bisphosphonate, carboxylate, and catechol). In this embodiment, the embolic hydrogel may further comprise a solution of metal ions or metal nanoparticles (e.g., gold, silver, copper, iron, calcium, magnesium, and zinc), in which case, the metal coordinating ligand and the metal ions or metal nanoparticles may interact with each other to form the primary reversible crosslinks within the polymer backbone of the single functionalized polymer.

The embolic hydrogel further comprises at least one phase-transitioning polymer that creates secondary crosslinks (e.g., physical crosslinks) therein in response to a physiological environment (e.g., in response to a change in one or more of a pH, temperature, and an ionic content induced by the physiological environment), such that the embolic hydrogel transitions from the gel state to a solid state. In one example, the embolic hydrogel transitions from the gel state to the solid state after ten seconds in response to the physiological environment. In one embodiment, one of the phase-transitioning polymer comprises the polymer backbone of one of functionalized polymer(s). In this embodiment, the functionalized polymer(s) may comprise a plurality of functionalized polymers, the phase-transitioning polymer(s) may comprises a single phase-transitioning polymer comprising the polymer backbone of only one of the functionalized polymers. In another embodiment, one or more of the phase-transitioning polymer(s) is not chemically modified with any of the functional group(s), such that phase-transitioning polymer does not contribute to the formation of the primary reversible crosslinks on the polymer backbone(s).

Any polymer backbone that is not composed of phase-transitioning polymer may, e.g., comprise polyethylene glycol (PEG), polyvinyl alcohol, gelatin, polyacrylamide, poly acrylic acid, poly(n-vinyl pyrrolidone) (PVP), poly(2-hydroxyethyl methacrylate) (pHEMA), poly(glycerol monomethacrylate) (pGMA), poly(ethylene glycol) methacrylate (pEGMA), poly(dimethylacrylamide) (pDMAAm), or polyacrylamide. In contrast, the phase-transitioning polymer may comprise alginate, pectins, methoxypectins, gellan gum, chitosan, carboxymethyl cellulose, block copolymers, carrageenan, agar, fucoidan, ulvan, hyaluronan and other exopolysaccharides and sulfonated polysaccharides, elastin-like proteins, poly(N-isopropylacrylamide), poloxamers, methylcellulose, or xyloglucan.

In one embodiment, the embolic hydrogel has a first viscosity when in the gel state, a second viscosity less than the first viscosity when in the viscous liquid state, and a third viscosity greater than the first viscosity when in the solid state. For example, the first viscosity may be in the range of 1 N·s/m²-1000N·s/m², the second viscosity may be in the range of 0.0001N·s/m²-10N·s/m², and the third viscosity may be in the range of 500N·s/m²-100000N·s/m². In another embodiment, the embolic hydrogel has a first storage modulus when in the gel state, a second storage modulus less than the first storage modulus when in the viscous liquid state, and a third storage modulus greater than the first storage modulus when in the solid state. For example, first storage modulus may be in the range of 1 Pa-500 Pa, the second storage modulus may be in the range of 0.01 Pa-10 Pa, and the third storage modulus may be in the range of 50 Pa-5000 Pa. In this embodiment, the embolic hydrogel may have a first loss modulus less than the first storage modulus when in the gel state, a second loss modulus greater than the second storage modulus when in the viscous liquid state, and a third loss modulus less than the third storage modulus when in the solid state. In still another embodiment, the embolic hydrogel, when in the viscous liquid state, is configured for being injected through a delivery catheter having an inner lumen equal to or less than 0.018″ in diameter at a force less than 50N, and preferably, less than 30N.

In an optional embodiment, the embolic hydrogel may further comprise a radiopacity agent (e.g., an iodine-based contrast agent or a metal-based contrast agent), a pharmacological agent (e.g., a chemotherapeutic pharmacological agent, an analgesic pharmacological agent, or an anesthetic pharmacological agent), and/or a radioisotope.

In accordance with another aspect of the present inventions, a method of treating a patient having a vascular defect (e.g., one of a hemorrhage, a hematoma, an arteriovenous malformation (AVM), an arteriovenous fistula, and an aneurysm). The vascular defect may be located in the cerebral vasculature of the patient.

The method comprises injecting the embolic hydrogel through a medical device (e.g., a delivery catheter), such that the embolic hydrogel transitions from a gel state to a viscous liquid state during injection through the medical device, and delivering the injected embolic hydrogel (e.g., endovascularly) from the medical device into the patient at a target delivery site (e.g., a vascular target site), such that the embolic hydrogel transitions back from the viscous liquid state to the gel state (e.g., within fifteen seconds) when delivered from the medical device into the patient. The method further comprises exposing the delivered embolic hydrogel to a physiological environment within the patient at the target delivery site, such that the delivered embolic hydrogel transitions from the gel state into a solid state (e.g., after ten seconds) in response to a change in a physiological parameter (e.g., one of a pH, temperature, and ionic content) induced by the physiological environment, thereby treating the vascular defect.

In one method, the embolic hydrogel has a first viscosity when in the gel state, a second viscosity less than the first viscosity when in the viscous liquid state, and a third viscosity greater than the first viscosity when in the solid state. For example, the first viscosity may be in the range of 1 N·s/m²-1000N·s/m², the second viscosity may be in the range of 0.0001 N·s/m²-1N·s/m², and the third viscosity may be in the range of 1000 N·s/m²-100000N·s/m². In another method, the embolic hydrogel has a first storage modulus when in the gel state, a second storage modulus less than the first storage modulus when in the viscous liquid state, and a third storage modulus greater than the first storage modulus when in the solid state. For example, the first storage modulus may be in the range of 5 Pa-500 Pa, the second storage modulus may be in the range of 0.01 Pa-5 Pa, and the third storage modulus may be in the range of 50 Pa-5000 Pa. In this method, the embolic hydrogel may have a first loss modulus less than the first storage modulus when in the gel state, a second loss modulus greater than the second storage modulus when in the viscous liquid state, and a third loss modulus less than the third storage modulus when in the solid state. In another method the embolic hydrogel is injected through an inner lumen of the method device equal to or less than 0.018″ in diameter at a force less than 50N, and preferably less than 30N.

An optional method further comprises medically imaging the embolic hydrogel when in the solid state. Another optional method further comprises delivering pharmacological therapy or radio therapy to the target delivery site via the embolic hydrogel.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of preferred embodiments of the present invention, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated. In order to better appreciate how the above-recited and the other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a diagram illustrating one embodiment of an embolic hydrogel in accordance with the present inventions, particularly showing the embolic hydrogel undergoing a dual-stage crosslinking process;

FIG. 2 is a diagram illustrating another embodiment of an embolic hydrogel in accordance with the present inventions, particularly showing the embolic hydrogel undergoing a dual-stage crosslinking process;

FIG. 3 is a diagram illustrating still another embodiment of an embolic hydrogel in accordance with the present inventions, particularly showing the embolic hydrogel undergoing a dual-stage crosslinking process;

FIG. 4 is a diagram illustrating yet another embodiment of an embolic hydrogel in accordance with the present inventions, particularly showing the embolic hydrogel undergoing a dual-stage crosslinking process;

FIG. 5 is a diagram illustrating yet another embodiment of an embolic hydrogel in accordance with the present inventions, particularly showing the embolic hydrogel undergoing a dual-stage crosslinking process;

FIG. 6 is a diagram illustrating yet another embodiment of an embolic hydrogel in accordance with the present inventions, particularly showing the embolic hydrogel undergoing a dual-stage crosslinking process;

FIG. 7 is a specific example of the embolic hydrogel illustrated in FIG. 1, particularly showing primary reversible crosslinks created between two functionalized polymers;

FIG. 8A is a plot of measured viscosity versus shear rate applied to a synthesized formulation A of an embolic hydrogel when in a gel state and a viscous liquid state;

FIG. 8B is a plot of a measured storage modulus and measured loss modulus versus strain applied to the synthesized formulation A of the embolic hydrogel when in the gel state;

FIG. 8C is a plot of a measured storage modulus and measured loss modulus versus time during a cycling between a relatively low strain and a relatively high strain applied to the synthesized formulation A of the embolic hydrogel;

FIG. 8D is a plot of a measured storage modulus and measured loss modulus versus strain applied to the synthesized formulation A of the embolic hydrogel when in a solid state;

FIG. 9A is a plot of measured viscosity versus shear rate applied to a synthesized formulation B of an embolic hydrogel when in a gel state and a viscous liquid state;

FIG. 9B is a plot of a measured storage modulus and measured loss modulus versus strain applied to the synthesized formulation B of the embolic hydrogel when in the gel state;

FIG. 9C is a plot of a measured storage modulus and measured loss modulus versus time during a cycling between a relatively low strain and a relatively high strain applied to the synthesized formulation B of the embolic hydrogel;

FIG. 9D is a plot of a measured storage modulus and measured loss modulus versus strain applied to the synthesized formulation B of the embolic hydrogel when in a solid state;

FIG. 10 is a flow diagram illustrating one method of delivering embolic hydrogel into the vasculature of a patient to treat a cerebral hematoma; and

FIGS. 11A-11G are plan views illustrating the treatment of the cerebral hematoma in accordance with the method illustrated in FIG. 10.

### DETAILED DESCRIPTION OF THE ILLUSTRATED EMBODIMENTS

The embolic materials disclosed herein can be quickly and efficiently delivered through a conventional small diameter delivery catheter (e.g., a microcatheter) to a vascular target site in a very controlled manner, while also effectively and consistently occluding the flow of blood at the vascular target site. In particular, various embolic hydrogels are described herein that utilize dual-stage crosslinking, wherein a first reversible (self-healing) crosslinking stage facilitates shear-thinning of the embolic hydrogel for injection (e.g., via a syringe) through the small diameter delivery catheter to the vascular target site, and a second crosslinking stage facilitates solidification or stiffening of the embolic hydrogel only after it has been delivered to the vascular target site. The use of dual-stage crosslinking allows for better control of the pre-delivery, trans-delivery, and post-delivery of the viscoelastic properties and in-situ transition kinetics of the embolic hydrogels.

In this manner, the embolic hydrogels described herein may be pre-mixed and then injected at a reasonable force (e.g., lower than 50N, and preferably lower than 30N) through a delivery catheter having relatively small inner lumens, e.g., 0.025″, or even 0.018″ or less in diameter. That is, due to the primary reversible crosslinking stage, such embolic hydrogels transition from its relatively thick gel state to a relatively thin viscous liquid state when injected through delivery catheters, thereby facilitating injection of the embolic hydrogel through the delivery catheter, and then from its viscous liquid state back to its gel state when exiting from the delivery catheter into the patient, thereby providing the desired control of the embolic hydrogels in situ. Due to secondary crosslinking stage, such embolic hydrogels also further solidify to occlude the vascular target site only in the presence of a physiological environment (i.e., in situ), and thus, will not prematurely stiffen prior to its delivery to the vascular target site, and will do so without having to add crosslinking initiators during or after delivery of the embolic hydrogels. Thus, the embolic hydrogels described herein may be pre-mixed prior to injection through the delivery catheter, thereby ensuring that components of the embolic hydrogels are thoroughly and consistently combined, and thus, avoiding the need to mix multiple components at the time of injection and further minimizing deployment-to-deployment variation.

Such embolic hydrogels, in addition to comprising an aqueous buffer, comprise at least one functionalized polymer respectively having at least one polymer backbone chemically modified with one or more functional groups that facilitate formation of primary reversible crosslinks (e.g., covalent crosslinks) on the polymer backbone(s). As a result, the embolic hydrogel will transition from its gel state (e.g., prior to delivery through a delivery catheter into the patient) to its viscous liquid state in response to a shear force applied to the embolic hydrogel during injection (e.g., during delivery through the delivery catheter), and transition back to its gel state in response to an absence of shear forces (e.g., after delivery through the delivery catheter into the patient).

As an example, the embolic hydrogels described herein, when in their gel states, may have first viscosities in the range of 1N·s/m²-1000N·s/m² (measured at a relatively low shear rate of 0.1/s), and when in their viscous liquid states, may have second lower viscosities in the range of 0.0001N·s/m²-10N·s/m² (measured at a relatively high shear rate of 1000/s). As another example, the embolic hydrogels described herein, in their gel states, may have first storage moduli in the range of 1 Pa-500 Pa (measured at relatively low strain of 0.1%), and when in their viscous liquid states, a second lower storage moduli in the range of 0.01 Pa-10 Pa (measured at a relatively high strain of 1000%). The embolic hydrogels described herein, when in their gel states, will have storage moduli that are significantly greater than their loss moduli, and when in their viscous liquid states, will have storage moduli that are significantly less than their loss moduli.

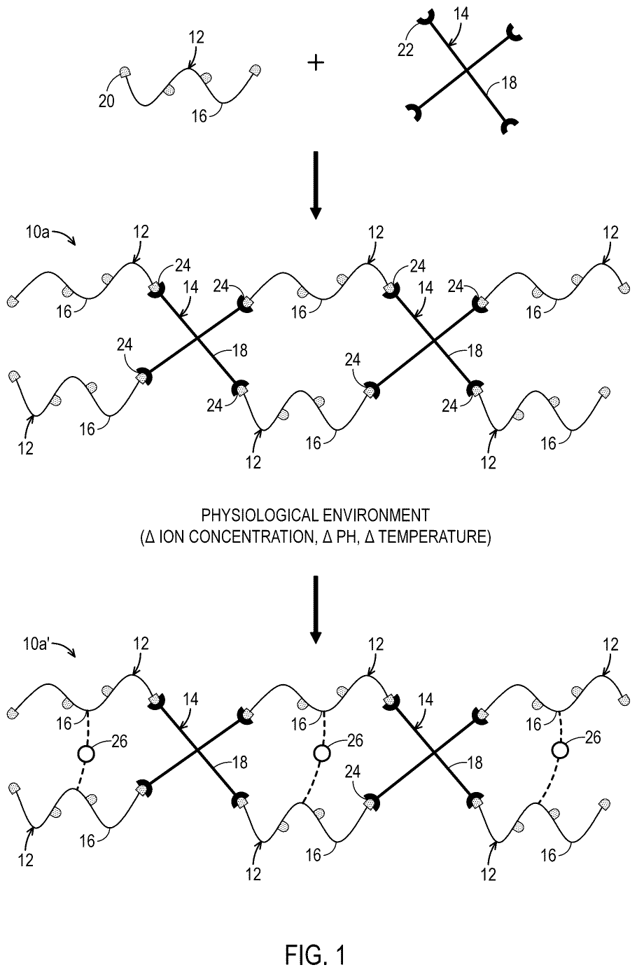

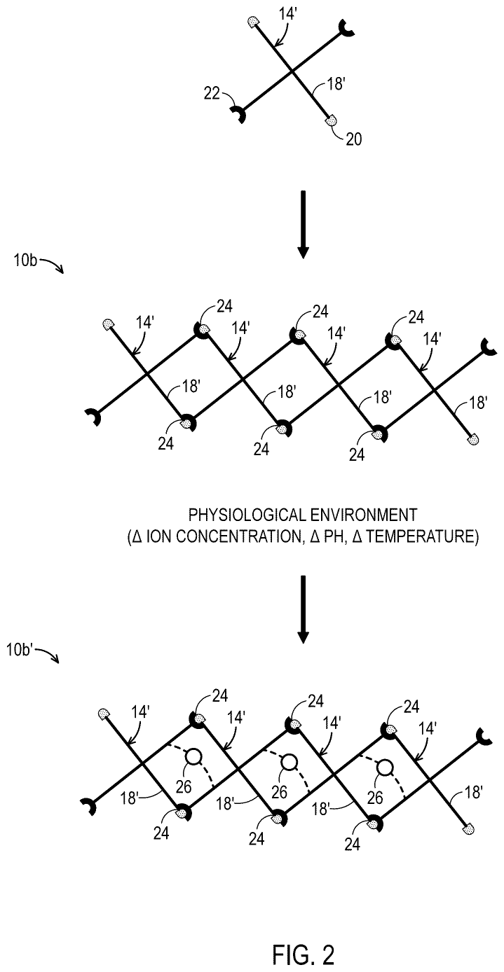

In one embodiment, the polymer backbone(s) of the functionalized polymer(s) may be chemically modified with at least two different functional groups that interact with each other to form the primary reversible crosslinks on the polymer backbone(s) of the functionalized polymer(s). Two functionalized polymers may be provided (e.g., in the embolic hydrogel 10a illustrated in FIG. 1), in which case, the polymer backbones of the two functionalized polymers may be chemically modified respectively with two of the different functional groups, which interact with each other to form the primary reversible crosslinks between the polymer backbones of the two functionalized polymers. Or a single functionalized polymer may be provided (e.g., in the embolic hydrogel 10b illustrated in FIG. 2), in which case, the polymer backbone of the single functionalized polymer may be chemically modified with the different functional groups, which interact with each other to form the primary reversible crosslinks within the polymer backbone of the single functionalized polymer.

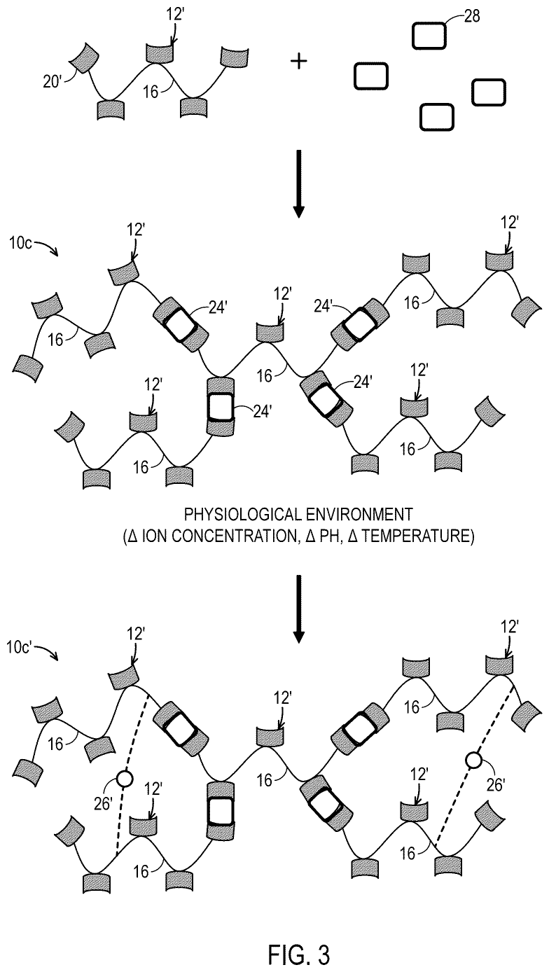

In another embodiment (e.g., the embolic hydrogel 10c illustrated in FIG. 3), one of the functional groups comprises a metal coordinating ligand, in which case, the embolic hydrogel may further comprise a solution of metal ions or metal nanoparticles that interact with the coordinating ligand to form the primary reversible crosslinks on the polymer backbone(s) of the functionalized polymer(s).

Significantly, the embolic hydrogels described herein also comprise one or more phase-transitioning polymers that create secondary crosslinks therein in response to a physiological environment, such that the embolic hydrogels transition from their gel states into solid states (e.g., after an elapsed time (e.g., greater than 10 seconds). As an example, the embolic hydrogels described herein, when in their solid states, may have third viscosities that are higher than their first viscosities when in their gel states, in the range of 500N·s/m²-100000N·s/m² (measured at a relatively low shear rate of 0.1/s). As another example, the embolic hydrogels described herein, when in their solid states, may have third storage moduli that are higher than their first storage moduli when in their gel states in the range of 50 Pa-5000 Pa (measured at a relatively low strain of 0.1%). The embolic hydrogels described herein, in their solid states, will have loss moduli that are significantly less than their storage moduli. The embolic hydrogels described herein are designed, such that the secondary crosslinks endure and do not transition back to the gel state as long as such embolic hydrogels remain in the physiological environment that induced the creation of the secondary crosslinks.

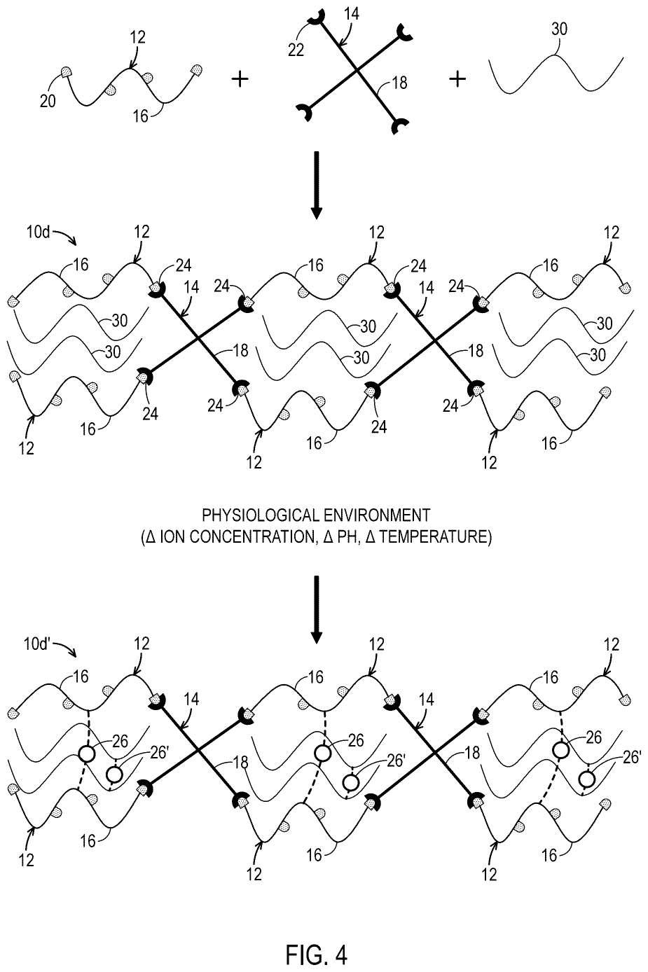

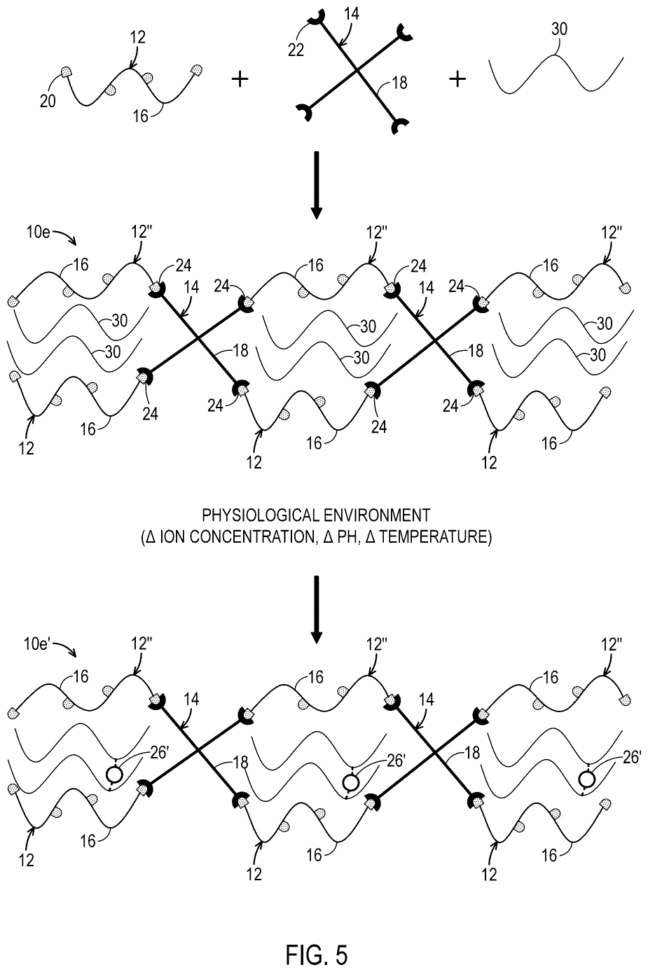

In one embodiment (e.g., the embolic hydrogels 10a-10c illustrated in FIGS. 1-3), one of the phase-transitioning polymer(s) comprises the polymer backbone of one of the functionalized polymer(s). In another embodiment, (e.g., the embolic hydrogels 10d-10e illustrated in FIGS. 4-5), one of the phase-transitioning polymer(s) may not be chemically modified with any of the functional groups, such that the phase-transitioning polymer does not contribute to the formation of the primary reversible crosslinks on the functionalized polymer(s), while enhancing the secondary crosslinks within the phase-transitioning polymer(s).

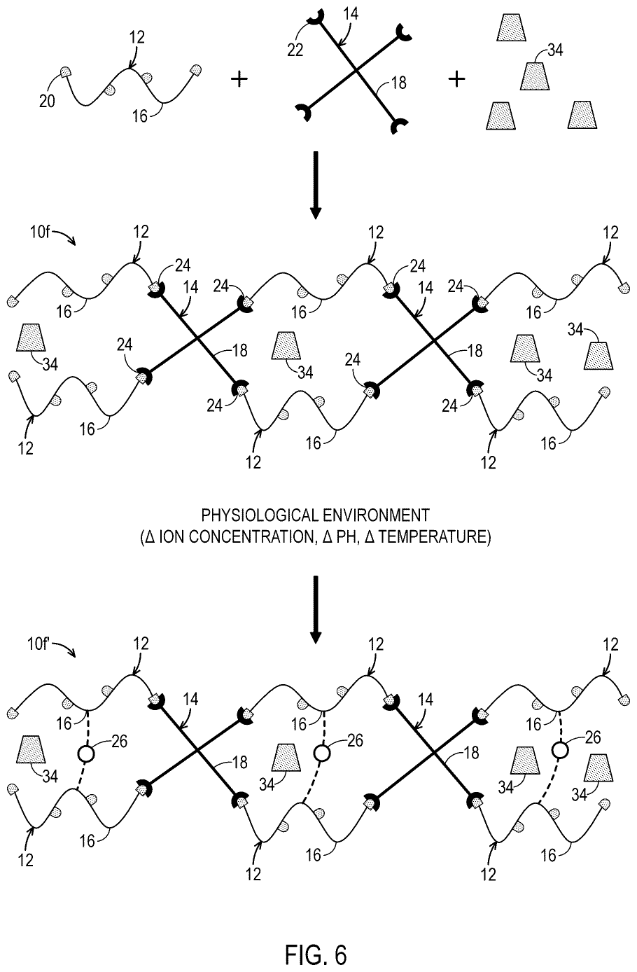

In optional embodiments (e.g., the embolic hydrogel 10f illustrated in FIG. 6), additional therapeutic and/or diagnostic agents may be added to the embolic hydrogels described herein.

It should be appreciated that, while the embolic hydrogels described herein lend themselves well to performing endovascular embolization in the brain of a patient via injection through conventional microcatheters, such embolic hydrogels may be used for endovascular embolization in more peripheral regions of the patients via injection through conventional microcatheters or larger diameter catheters, or may even be used to treat vascular or non-vascular defects or injuries via injection into target delivery sites within the interstitial spaces of the body of the patient (e.g., wound care).

Referring now to FIG. 1, one specific embodiment of an embolic hydrogel 10a will be described. As shown in the top diagram of FIG. 1, in addition to an aqueous buffer (e.g., phosphate buffered saline that has been pH adjusted to obtain the desired viscosity/shear properties for the embolic hydrogel 10a), a first functionalized polymer 12 and a second functionalized polymer 14 (only a monomer shown in the top diagram of FIG. 1) may be combined to create the embolic hydrogel 10a (shown in the center diagram of FIG. 1).

The first functionalized polymer 12 has a natural or synthetic polymer backbone 16, at least a portion of which has been chemically modified with a functional group 20, while the second functionalized polymer 14 similarly has a second natural or synthetic polymer backbone 18, at least a portion of which has been chemically modified with a functional group 22. Although, as illustrated in FIG. 1, both of the functionalized polymers 12, 14 are fully functionalized (i.e., 100% of the polymer backbones 16, 18 have been respectively chemically modified with the functional groups 20, 22), alternatively, one or both of the functionalized polymers 12, 14 may be partially functionalized (i.e., less than 100% (e.g., only 25% or 35%) of the polymer backbones 16, 18 may be respectively chemically modified with the functional groups 20, 22). In the illustrated embodiment, the polymer backbone 16 of the first functionalized polymer 14 has a linear structure, while the polymer backbone 18 of the second functionalized polymer 14 has a multi-arm structure. However, it should be appreciated that that either of the polymer backbones 16, 18 may, e.g., be a linear, branched, or multi-arm structure. The concentration of each of the first and second functionalized polymers 12, 14 in the embolic hydrogel 10a may be, e.g., in the range of 0.1 wt %- 4 wt %.

When the first functionalized polymer 12 and the second functionalized polymer 14 are combined to create the embolic hydrogel 10a, as shown in the center diagram of FIG. 1, the respective functional groups 20, 22 interact to create primary reversible crosslinks 24 between the polymer backbones 16, 18 of the functionalized polymers 12, 14, such that the embolic hydrogel 10a is shear thinning and reversible (i.e., thins from its gel state to its viscous liquid state in the presence of shear force, and then returns from its viscous liquid state back to its gel state in the absence of shear force).

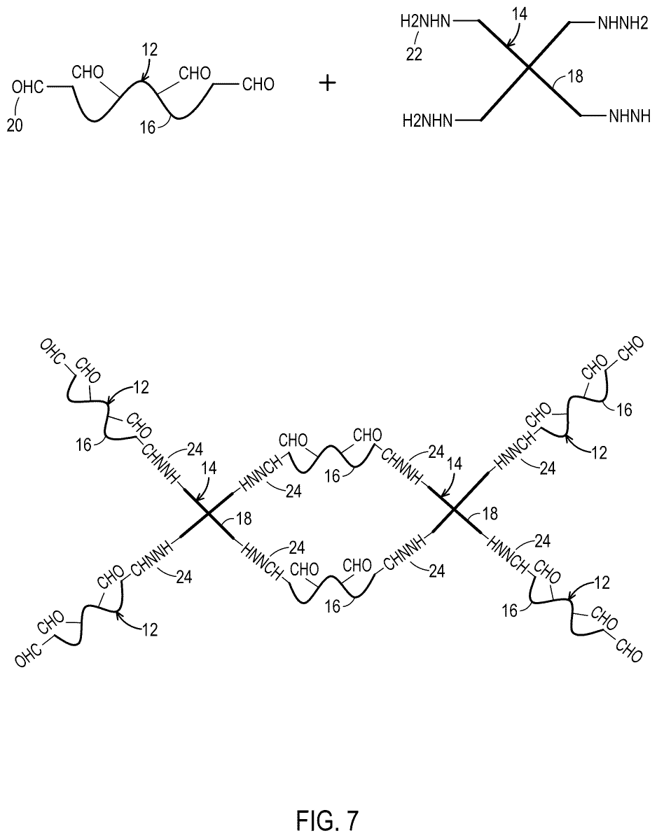

In one embodiment, the functional group 20 of the first functionalized polymer 12 comprises ketones, aldehydes, benzaldehydes, or derivatives thereof, and the functional group 22 of the second functionalized polymer 14 is a nucleophilic functional group, such as, e.g., amine, hydrazide, acylhydrazide, aminooxy, or derivatives thereof. The interaction between these functional groups will create Schiff-based covalent crosslinks. For example, as shown in FIG. 7, functional group 20 of the first functionalized polymer 12 comprises aldehyde, and the functional group 22 of the second functionalized polymer 14 comprises a hydrazide. As a result of the interaction between the functional groups 20, 22, nitrogen-carbon covalent bonds (primary reversible crosslinks 24) are created between the polymer backbones 16, 18 of the functionalized polymers 12, 14, as best shown in the bottom diagram of FIG. 7.

The polymer backbone 18 of the second functionalized polymer 14 may not be phase-transitioning and may, e.g., comprise of, but not be limited to, polyethylene glycol (PEG), polyvinyl alcohol, gelatin, poly acrylic acid, poly(n-vinyl pyrrolidone) (PVP), poly(2-hydroxyethyl methacrylate) (pHEMA), poly(glycerol monomethacrylate) (pGMA), poly(ethylene glycol) methacrylate (pEGMA), poly(dimethylacrylamide) (pDMAAm) and polyacrylamide. In contrast, as shown in the bottom diagram of FIG. 1, the polymer backbone 16 of the first functionalized polymer 12 is a phase-transitioning polymer backbone that creates secondary crosslinks 26 therein in response to a physiological environment, such that the embolic hydrogel 10a solidifies into a highly viscous (i.e., more dense) mass 10a′. Thus, when exposed to the physiological environment, the embolic hydrogel 10a will transition from its gel state to its solid state. As discussed in further detail below, the embolic hydrogel 10a will transition from its gel state to its solid state in response to a change in a physiological parameter (e.g., pH, temperature, or ion concentration) induced by the physiological environment to which the embolic hydrogel 10a is exposed. The phase-transitioning polymer backbone 16 may, e.g., comprise of, but not be limited to, alginate, pectins, methoxypectins, gellan gum, chitosan, carboxymethyl cellulose, block copolymers, carrageenan, agar, fucoidan, ulvan, hyaluronan and other exopolysaccharides and sulfonated polysaccharides, elastin-like proteins, poly(N-isopropylacrylamide), poloxamers, methylcellulose, and xyloglucan. In other embodiments, alternatively or in addition to, the polymer backbone 16 of the first functionalized polymer 12 being a phase-transitioning polymer backbone, the polymer backbone 18 of the second functionalized polymer 14 may be a phase-transitioning polymer backbone.

The particular mechanism for creating the secondary crosslinks 26 in the polymer backbone 16 of the first functionalized polymer 12 (and/or the polymer backbone 18 of the second functionalized polymer 14) will depend on the composition of the polymer backbones 16, 18. Such secondary crosslink mechanisms may be, e.g., physical crosslink formation, such as ion complex formation with functional groups, precipitation leading to increased polymer entanglement, and/or self-assembly of charged or hydrophobic domains.

As one example, selecting alginate, pectins, methoxypectins, or gellan gum for the polymer backbone 16 of the first functionalized polymer 12 will induce secondary crosslinks 26 in response to a change in ion concentration (e.g., by crosslinking with ions in the blood), as illustrated in the bottom diagram of FIG. 1. In particular, carboxylic acid or carboxyl groups in these types of polymer chains associate with the multivalent cations in the blood (including, but not limited, to, Ca²⁺, Mg²⁺, and Ba²⁺). Different polymer chains are able to associate with each ion, creating links between different polymer chains forming the crosslinked network.

As another example, selecting chitosan for the first polymer backbone 16 will induce secondary crosslinks 26 (e.g., via precipitation) in response to a change in pH. In particular, chitosan has amine groups along the polymer chain that allow for solubility at low pH. Once the chitosan polymer chain is exposed to a higher pH (>6.5), it becomes insoluble and precipitates to become more densely entangled leading to gel stiffening (higher modulus). Similarly, selecting carboxymethyl cellulose for the first polymer backbone 16 will induce secondary crosslinks 26 (e.g., via precipitation) in response to a change in pH. In particular, once the carboxymethyl cellulose polymer chain is exposed to a lower pH (<4), its charge is lowered, leading to precipitation to a gel via aggregation of multiple polymer chains.

As still another example, selecting block copolymers, elastin-like proteins, or poloxamers for the first polymer backbone 16 will induce secondary crosslinks 26 (via self-assembly) in response to a change in temperature, pH, or ionic content. In particular, block copolymer solutions containing polymer chains consisting of either hydrophobic and hydrophilic “blocks” or polycationic and polyanionic “blocks” can be designed, such that they change in solubility at different temperatures, pH, or ion concentration. Block copolymer solutions are soluble at one temperature, pH, or ion concentration, and then hydrophobic or charged blocks self-assemble to form a gel as these environmental conditions change. Elastin-like proteins containing polymer chains consisting of hydrophilic and hydrophobic blocks of amino acid residues are soluble below a critical solution temperature (e.g., below body temperature). As the temperature rises (e.g., to body temperature), the hydrophobic blocks become progressively less soluble and self-assemble to form a gel. Similarly, poloxamers containing polymer chains consisting of hydrophobic blocks of polyoxyethylene and hydrophilic blocks of polyoxypropylene are soluble below a critical solution temperature (e.g., below body temperature). As the temperature rises (e.g., to body temperature), the hydrophobic blocks become progressively less soluble and self-assemble to form a gel.

As yet another example, selecting pNIPAAM for the first polymer backbone 16 will induce secondary crosslinks 26 (via precipitation) in response to a change in temperature. In particular, PNIPAAM is soluble below a critical solution temperature (e.g., below body temperature). As the temperature rises (e.g., to body temperature), the PNIPAAM becomes insoluble and precipitates out of solution.

As yet another example, selecting methylcellulose for the first polymer backbone 16 will induce secondary crosslinks 26 (via precipitation) in response to a change in temperature. In particular, methylcellulose is hydrophilic at low temperatures (e.g., below body temperature) due to its ability to form hydrogen bonds with water. As the temperature rises (e.g., at body temperature), the polymer loses the hydrogen bonds to the water, rendering it hydrophobic, such that it precipitates.

As yet another example, selecting xyloglucan for the first polymer backbone 16 will induce secondary crosslinks (e.g., via self-assembly) in response to a change in temperature. In particular, a portion of galactos residue (e.g., >35%) may be removed from the xyloglucan, thereby rendering it thermoresponsive. The hydrophilic portion of the chemically modified xyloglucan self-assemble in response to an increase in temperature.

In some embodiments, certain phase-transitioning polymers (e.g., chitosan or pNIPAAM) may form increased entanglements that occlude the vessel, thereby serves as a hemostatic agent for promotion of coagulation when exposed to blood.

Notably, the use of two separate functionalized polymers 12, 14 facilitates the independent control of the ratio the functional groups 16, 18 relative to each other when combining the functionalized polymers 12, 14, such that the viscoelastic properties of the embolic hydrogel 10a, in its gel state, viscous liquid state, and solid state, may be more effectively tuned. Although the embolic hydrogel 10a illustrated in FIG. 1 comprises multiple functionalized polymers, and in particular, two mono-functionalized polymers 12, 14 (i.e., the polymer backbone of each functionalized polymer is chemically modified with only one functional group), a hetero-functionalized polymer may be used, e.g., the polymer backbone of one or both of the functionalized polymers 12, 14 may be functionalized with multiple different functional groups.

For example, referring now to FIG. 2, another specific embodiment of an embolic hydrogel 10b will be described. As shown in the top diagram of FIG. 2, an aqueous buffer (e.g., pH-adjusted phosphate buffered saline) may be combined with a single hetero-functionalized polymer 14′ to create the embolic hydrogel 10b (shown in the center diagram of FIG. 2). The hetero-functionalized polymer 14′ differs from the homo-functionalized polymer 14 described above with respect to FIG. 1, with the exception that a polymer backbone 18′ of the hetero-functionalized polymer 14′ is chemically modified with multiple different functional groups, and in this case, the two functional groups 20, 22. In this case, the ratio of the functional groups 20, 22 relative to each other can be selected during the synthesis of the hetero-functionalized polymer 14′, which may be more difficult to accomplish than selecting the ratio of the functional groups 20, 22 when combining two homo-functionalized polymers after they have been synthesized. Notably, the multi-arm structure of the polymer backbone 16′ facilitates its chemical modification with multiple functional groups by providing more structural elements on which the functional groups 20, 22 may attach, although in alternative embodiments, the polymer backbone 16′ may have a linear structure.

As shown in the center diagram of FIG. 2, the functional groups 20, 22 interact to create primary reversible crosslinks 24 within the polymer backbone 16′ of the functionalized polymer 12′, such that the embolic hydrogel 10b is shear thinning and self-healing (i.e., thins from its gel state to its viscous liquid state in the presence of shear force, and then returns from its viscous liquid state back to its gel state in the absence of shear force). The polymer backbone 18′ of the hetero-functionalized polymer 14′ also differs from the polymer backbone 18 of the homo-functionalized polymer 14 described above with respect to FIG. 1, in that it is a phase-transitioning polymer that creates secondary crosslinks 26 therein in response to a physiological environment, such that the embolic hydrogel 10b solidifies into a highly viscous mass 10b′, as illustrated in the bottom diagram of FIG. 2. Thus, when exposed to the physiological environment, the embolic hydrogel 10b will transition from its gel state to its solid state.

Referring now to FIG. 3, another still specific embodiment of an embolic hydrogel 10c will be described. As shown in the top diagram of FIG. 3, in addition to an aqueous buffer (e.g., pH-adjusted phosphate buffered saline), a functionalized polymer 12′ having the phase-transitioning polymer backbone 16, at least a portion of which has been chemically modified with a functional group, and in particular a metal coordinating ligand 20′, and a metallic solution 28 (e.g., one containing metallic ions or nano-particles), may be combined to create the embolic hydrogel 10c (shown in the center diagram of FIG. 3). The concentration of the metallic solution 28 may be, e.g., in the range of 0.1 mM to 100 mM. The metallic solution 28 may, e.g., comprise of, but not limited to gold, silver, copper, iron, calcium, magnesium, or zinc.

When the first functionalized polymer 12′ and the metallic solution 28 are combined to create the embolic hydrogel 10b, as shown in the center diagram of FIG. 3, the respective metal coordinating ligand 20′ and the metallic ions or nano-particles of the metallic solution 28 interact to create primary reversible crosslinks 24′ between the polymer backbone 16 of the functionalized polymer 12′ (i.e., via the metal coordinating ligand 20′ and the selected metal element of the metallic solution 28), such that the embolic hydrogel 10c is shear thinning and self-healing (i.e., thins from its gel state to its viscous liquid state in the presence of shear force, and then returns from its viscous liquid state back to its gel state in the absence of shear force). The interaction between the metal coordinating ligand 20′ and the metallic ions or nano-particles in the metallic solution 28 will create metallic crosslinks.

As previously discussed above, the polymer backbone 16 of the first functionalized polymer 12 is a phase-transitioning polymer backbone that creates secondary crosslinks 26 therein in response to a physiological environment, such that the embolic hydrogel 10c solidifies into a highly viscous mass 10c′, as illustrated in the bottom diagram of FIG. 3. Thus, when exposed to the physiological environment, the embolic hydrogel 10c will transition from its gel state to its solid state.

In an optional embodiment, any of the embolic hydrogels 10a-10c respectively illustrated in FIGS. 1-3 may comprise additional non-functionalized phase-transitioning polymer (dopant), such that the secondary crosslinking of the embolic hydrogels may be enhanced without affecting the primary reversible crosslinking of the embolic hydrogels. For example, as illustrated in FIG. 4, one embodiment of an embolic hydrogel 10d is similar to the embolic hydrogel 10a illustrated in FIG. 1 in that it comprises the first and second functionalized polymers 12, 14. However, the embolic hydrogel 10d differs from the embolic hydrogel 10a in that it additionally comprises non-functionalized phase-transitioning polymer 30.

The non-functionalized phase-transitioning polymer 30 may have the same composition as either of the polymer backbones 16, 18 of the functionalized polymers 12, 14 or may have a composition that differs from that of both of the polymer backbones 16, 18 of the functionalized polymers 12, 14. As shown in the center diagram of FIG. 4, the non-functionalized phase-transitioning polymer 30 does not contribute to the creation of primary reversible crosslinks 24 between the polymer backbones 16, 18. As shown in the bottom diagram of FIG. 4, the non-functionalized phase-transitioning polymer 30 creates additional secondary links 26′ within the non-functionalized phase-transitioning polymer 30 in response to a physiological environment. Although the mechanisms that create the secondary links 26, 26′ are illustrated as being the same, such mechanisms may be different. Also, although not shown, the secondary links 26′ may also be created between the polymer backbone 16 of the first functionalized polymer 12 and the non-functionalized phase-transitioning polymer 30, assuming that the polymer backbone 16 of the first functionalized polymer 12 and the non-functionalized phase-transitioning polymer 30 either comprise the same material or the particular mechanism for creating the secondary crosslinks 26′ involves precipitation. In this case, the network created by the primary reversible crosslinks 24 between functionalized polymers 12 and 14 forms an interpenetrating network with the physical crosslinked network formed by the non-functionalized phase-transitioning polymer 30 and itself, leading to gel stiffening.

In another optional embodiment, none of the functionalized polymers 12, 14, 14′ in the embolic hydrogels 10a-10c respectively illustrated in FIGS. 1-3 are phase-transitioning polymers, but instead, a separate non-functionalized phase-transitioning polymer is used. In this manner, the primary transient crosslinks 24 and secondary crosslinks 26 (i.e., the viscoelastic properties of the embolic hydrogel during its gel state, its viscous liquid state, and its solid state) may be independently tuned or optimized.

For example, as illustrated in FIG. 5, an embolic hydrogel 10e is similar to the embolic hydrogel 10a illustrated in FIG. 1 in that it comprises the first and second functionalized polymers 12″, 14. However, the embolic hydrogel 10e differs from the embolic hydrogel 10a in that the first functionalized polymer 12″ is not a phase-transitioning polymer. Like the polymer backbone 18 of the second functionalized polymer 14, the polymer backbone 16 of the first functionalized polymer 12″ may, e.g., comprise of, but not be limited to, polyethylene glycol (PEG), polyvinyl alcohol, gelatin, or polyacrylamide. The embolic hydrogel 10e additionally comprises non-functionalized phase-transitioning polymer 30 (dopant) having a polymer backbone 32 that is separate from the polymer backbone 16 of the first functionalized polymer 12″ (as well as from the polymer backbone 18 of the second functionalized polymer 14).

As shown in the center diagram of FIG. 5, the non-functionalized phase-transitioning polymer 30 does not contribute to the creation of primary reversible crosslinks 24 between the polymer backbones 16, 18 of the functionalized polymers 12, 14. However, as shown in the bottom diagram of FIG. 5, the non-functionalized phase-transitioning polymer 30 creates additional secondary links 26′ within the polymer backbone 32, while no secondary links are created within the polymer backbone 16 of the first functionalized polymer 12. In this case, the network created by the primary reversible crosslinks 24 between functionalized polymers 12 and 14 forms an interpenetrating network with the network of secondary crosslinks 26′ formed by polymer backbone 32 of the non-functionalized phase-transitioning polymer 30 and itself, leading to gel stiffening.

In yet another embodiment, any of the embolic hydrogels 10a-10e described above in FIGS. 1-5 may further comprise a pharmacological agent of interest, e.g., a chemotherapeutic pharmacological agent, an analgesic pharmacological agent, an anesthetic pharmacological agent, etc., or a radioisotope for selective localized delivery or radiation at the vascular target site. For example, as illustrated in FIG. 6, an embolic hydrogel 10f is similar to the embolic hydrogel 10a illustrated in FIG. 1 in that it comprises the first and second functionalized polymers 12, 14. However, the embolic hydrogel 10e differs from the embolic hydrogel 10a in that it further comprises a pharmacological agent of interest or a radioisotope 34.

In still another optional embodiment, any of the embolic hydrogels 10a-10e described above in FIGS. 1-5 may further comprise a radiopaque agent, e.g., iodine-based contrast agents (e.g., iohexol) or metal-based contrast agents (e.g., micro-scale or nano-scale metal powder, such as tantalum, bismuth trioxide, gold, tungsten, platinum, zirconium oxide, barium sulfate, tantalum oxide, bismuth subcarbonate, gadolinium or ytterbium). In this manner, the embolic hydrogels 10a-10d may be easily visualized under fluoroscopy. The concentration of radiopaque agent in the embolic hydrogel may be, e.g., in the range of 10 wt %-40 wt %.

A working example of an embolic hydrogel (Formulation A) corresponding to the embolic hydrogel 10d illustrated in FIG. 4 has been synthesized in the laboratory. This embolic hydrogel consists of aldehyde modified alginate (20% functionalization), 4-arm Poly(ethylene glycol) (10 kDa) modified with hydrazide, chemically unmodified alginate, tantalum powder (as contrast agent), and pH-adjusted phosphate-buffered saline. The concentration of aldehyde modified alginate is 0.32 wt %, the concentration of the hydrazide modified Poly(ethylene glycol) is 0.59 wt %, the concentration of chemically unmodified alginate is 0.48 wt %, the concentration of tantalum powder is 30 wt %, and the concentration of the phosphate-buffered saline is 68.6 wt %.

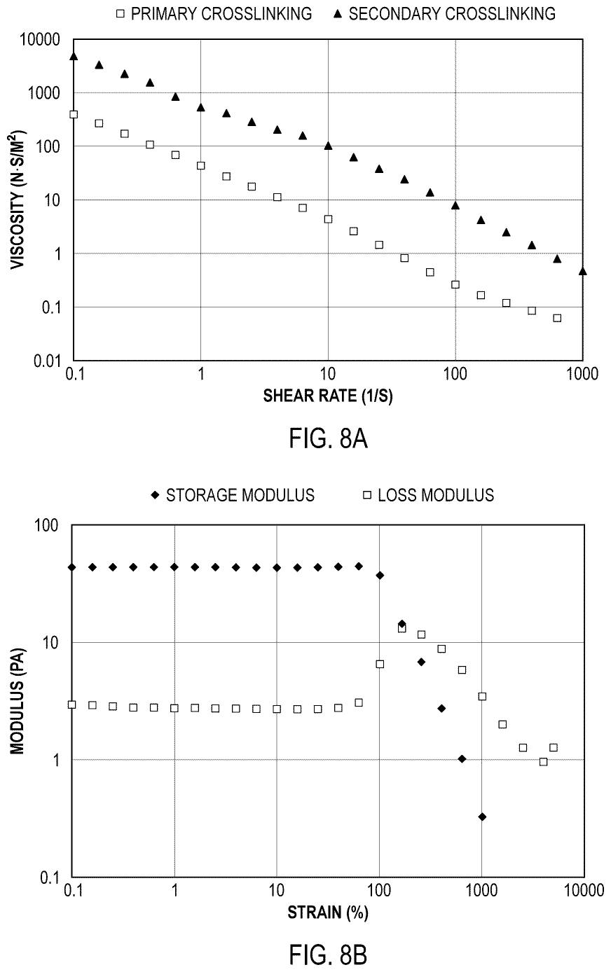

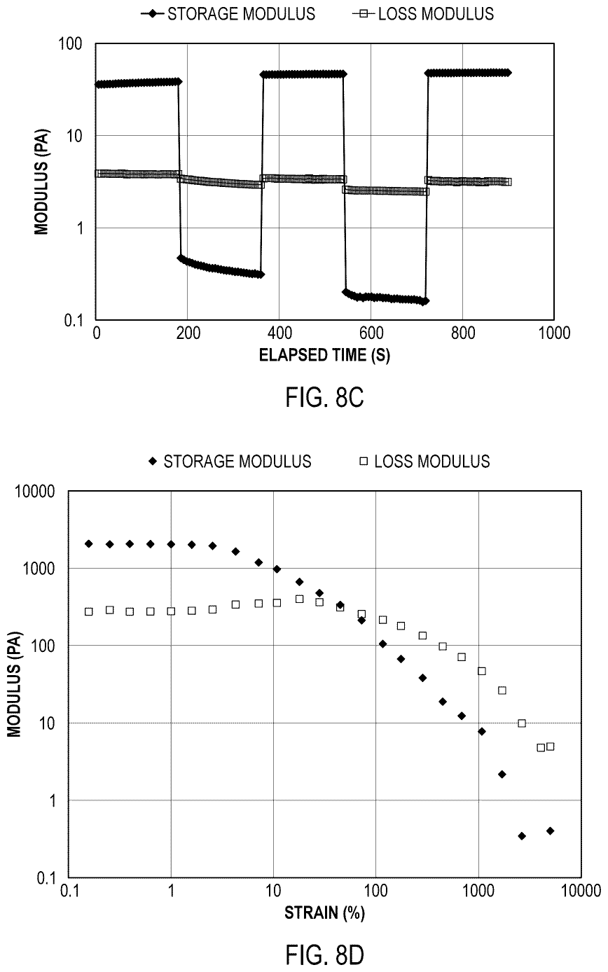

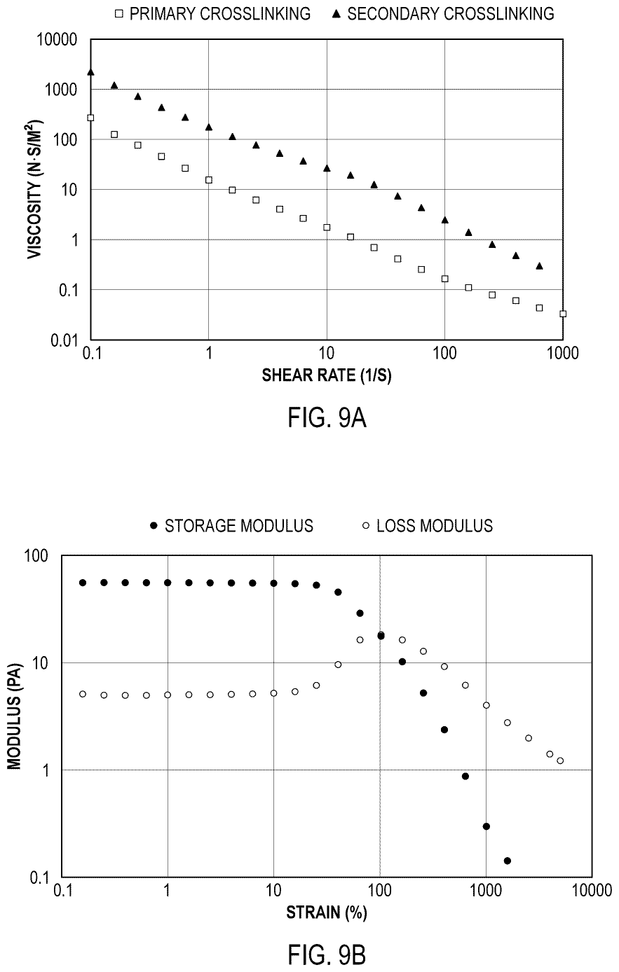

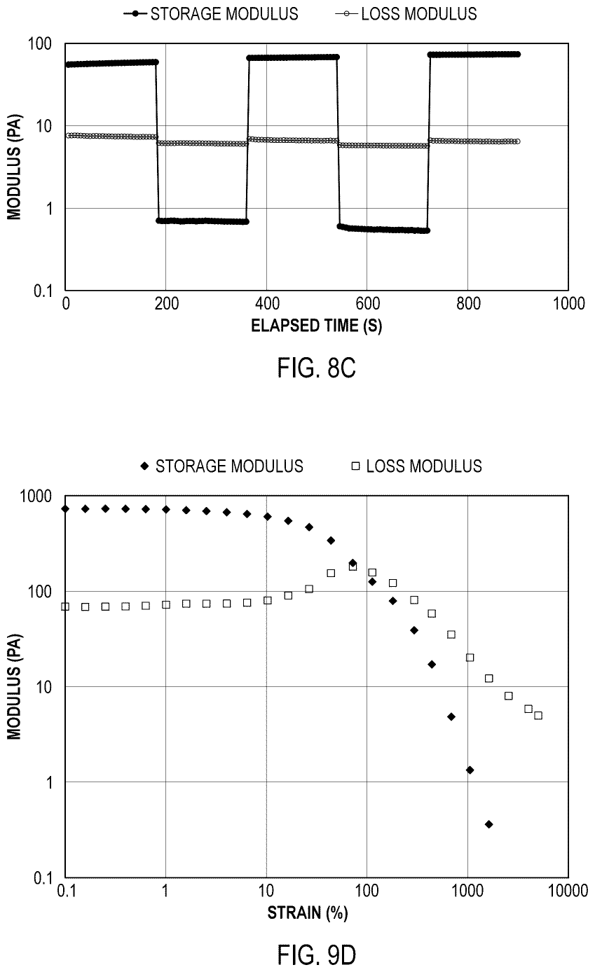

It has been proven that Formulation A of the embolic hydrogel satisfies the preferred ranges of the viscoelastic properties set forth above for facilitating the transition from its gel state to its highly viscous state. In particular, as illustrated in FIG. 8A-8B, the primary crosslinking stage (i.e., shear thinning) of Formulation A was tested by performing a shear rate sweep in the range of 0.1/s-10,000/s on Formulation A while its viscosity was measured, and performing a strain sweep in the range of 0.1%-10,000% on Formulation A while its storage modulus and loss modulus were measured.

As shown in FIG. 8A, the viscosity of Formulation A is approximately 400N·s/m² at the relatively low shear rate of 0.1/s and approximately 0.05 N·s/m² at the relatively high shear rate of 1000/s. As shown in FIG. 8B, the storage modulus and loss modulus of Formulation A are respectively approximately 40 Pa and 3 Pa at the relatively low strain of 0.1%, and respectively approximately 0.3 Pa and 3 Pa at the relatively high strain of 1000%. As can be appreciated, the storage modulus is significantly greater than the loss modulus when Formulation A is subjected to a relatively low strain 0.1%, while the loss modulus is significantly greater than the storage modulus when Formulation A is subjected to a relatively high strain 1000%. As can also be appreciated from FIG. 8B that the cross-over point between the storage modulus and the loss modulus of Formulation A, which is the point at which Formulation A transitions between a gel state and a viscous liquid state, occurs approximately at a strain of 150%.

A force injection test was also performed on Formulation A to test its injectability through a small diameter catheter. In particular, the force required to inject Formulation A through a catheter having a 0.0165″ diameter lumen was determined to be less than 30N. It can be appreciated that the force required to inject Formulation A through a catheter with a larger diameter lumen (e.g., 0.018″ or 0.025″) would be even less than that required to inject Formulation A through the catheter with the 0.0165″ diameter lumen.

It has also been proven that Formulation A is reversible or self-healing for facilitating the transition from its highly viscous state back to its gel state. In particular, as illustrated in FIG. 8C, the self-healing of Formulation A was tested by repeatedly cycling Formulation A between a 1% strain and a 1000% strain, while its storage modulus and the loss modulus were measured. As shown, the storage modulus of Formulation A consistently transitions between approximately 40 Pa (at 1% strain) and approximately 0.3 Pa (at 1000% strain), and the loss modulus of Formulation A is approximately 3 Pa (at both 1% strain and 1000% strain).

It has also been proven that Formulation A of the embolic hydrogel satisfies the preferred ranges of the viscoelastic properties set forth above for facilitating the transition from its gel state to its solid state. For example, as illustrated in FIGS. 8A and 8D, the second crosslinking stage (i.e., stiffening) of Formulation A was tested by submerging Formulation A into a solution of 10 mM of Calcium Chloride (CaCl₂) for 15 minutes (to emulate a physiological environment in a blood vessel), and after the required period of time for stiffening of the Formulation A, performing a shear rate sweep in the range of 0.1/s-10,000/s on submerged Formulation A while its viscosity was measured, and performing a strain sweep in the range of 0.1%-10,000% on submerged Formulation A while its storage modulus and loss modulus were measured. As shown in FIG. 8A, the viscosity of submerged Formulation A is approximately 6000N·s/m² at the relatively low shear rate of 0.1/s. As shown in FIG. 8D, the storage modulus of Formulation A is approximately 2000 Pa and 300 Pa at the relatively low strain of 0.1%.

Another working example of an embolic hydrogel (Formulation B) corresponding to the embolic hydrogel 10d illustrated in FIG. 4 has been synthesized in the laboratory. This embolic hydrogel consists of aldehyde modified alginate (20% functionalization), 4-arm Poly(ethylene glycol) (20 kDa) modified with hydrazide, chemically unmodified alginate, and tantalum powder (as contrast agent), and pH-adjusted phosphate-buffered saline. The concentration of aldehyde modified alginate is 0.39 wt %, the concentration of the hydrazide modified Poly(ethylene glycol) is 0.54 wt %, the concentration of chemically unmodified alginate is 0.43 wt %, the concentration of tantalum powder is 30 wt %, and the concentration of the phosphate-buffered saline is 68.6 wt %.

It has been proven that Formulation B of the embolic hydrogel satisfies the preferred ranges of the viscoelastic properties set forth above for facilitating the transition from its gel state to its highly viscous state. In particular, as illustrated in FIG. 9A-9B, the primary crosslinking stage (i.e., shear thinning) of Formulation B was tested by performing a shear rate sweep in the range of 0.1/s-10,000/s on Formulation B while its viscosity was measured, and performing a strain sweep in the range of 0.1%-10,000% on Formulation B while its storage modulus and loss modulus were measured.

As shown in FIG. 9A, the viscosity of Formulation B is approximately 900N·s/m² at the relatively low shear rate of 0.1/s and approximately 0.01 N·s/m² at the relatively high shear rate of 1000/s. As shown in FIG. 9B, the storage modulus and loss modulus of Formulation B are respectively approximately 60 Pa and 5 Pa at the relatively low strain of 0.1%, and respectively approximately 0.3 Pa and 5 Pa at the relatively high strain of 1000%. As can be appreciated, the storage modulus is significantly greater than the loss modulus when Formulation B is subjected to a relatively low strain 0.1%, while the loss modulus is significantly greater than the storage modulus when Formulation B is subjected to a relatively high strain 1000%. As can also be appreciated from FIG. 9B that the cross-over point between the storage modulus and the loss modulus of Formulation B is approximately at a strain of 250%.

A force injection test was also performed on Formulation B to test its injectability through a small diameter catheter. In particular, the force required to inject Formulation B through a catheter having a 0.0165″ diameter lumen was determined to be less than 30N. It can be appreciated that the force required to inject Formulation B through a catheter with a larger diameter lumen (e.g., 0.018″ or 0.025″) would be even less than that required to inject Formulation B through the catheter with the 0.0165″ diameter lumen.

It has also been proven that Formulation B is reversible or self-healing for facilitating the transition from its highly viscous state back to its gel state. In particular, as illustrated in FIG. 9C, the self-healing of Formulation B was tested by repeatedly cycling Formulation B between a 1% strain and a 1000% strain, while its storage modulus and the loss modulus were measured. As shown, the storage modulus of Formulation B consistently transitions between approximately 60 Pa (at 1% strain) and approximately 0.8 Pa (at 1000% strain), and the loss modulus of Formulation B is approximately 8 Pa (at both 1% strain and 1000% strain).

It has also been proven that Formulation B of the embolic hydrogel satisfies the preferred ranges of the viscoelastic properties set forth above for facilitating the transition from its gel state to its solid state. For example, as illustrated in FIGS. 9A and 9D, the second crosslinking stage (i.e., stiffening) of Formulation B was tested by submerging Formulation B into a Calcium Chloride (CaCl₂) solution, and after the required period of time for stiffening of the Formulation B, performing a shear rate sweep in the range of 0.1/s-10,000/s on submerged Formulation B while its viscosity was measured, and performing a strain sweep in the range of 0.1%-10,000% on submerged Formulation B while its storage modulus and loss modulus were measured. As shown in FIG. 9A, the viscosity of submerged Formulation B is approximately 2000N·s/m² at the relatively low shear rate of 0.1/s. As shown in FIG. 9D, the storage modulus and loss modulus of Formulation B are approximately 800 Pa and 80 Pa at the relatively low strain of 0.1%.

It should be appreciated that any of the embolic hydrogels 10a-10f described above may be supplied to a clinician as either a fully-hydrated ready-to-deploy form (i.e., with the aqueous buffer) or may be supplied to the clinician in a dried powder form (i.e., without the aqueous buffer), in which case, the powdered embolic hydrogel may be reconstituted into gel form by adding an aqueous solution by the clinician immediately prior to deployment within the patient. In this manner, the embolic hydrogel may have an increased shelf-life, a more simplified shipping and storage, and save time and cost. In one embodiment, any of the embolic hydrogels 10a-10f may be fabricated by first mixing the polymers and/or metallic solution together with an aqueous buffer into a gel form. Then, using common methods including lyophilization (sublimation), heat (evaporation), vacuum, spray drying, freeze drying, etc., the water may be removed from the gel form of the embolic hydrogel, resulting in a powdered embolic hydrogel. In one technique, all of the substances needed or desirable in a ready-to-deploy embolic hydrogel, such as salts, acids, bases, other adjuvants, or water soluble contrast agents and/or radiopaque agents, etc., may be included within an aqueous buffer that is then mixed with the polymers and/or metallic solution into the gel form prior to removal of the water. In this case, the reconstitution solution subsequently added to the powdered embolic hydrogel during the rehydration step may consist of only deionized water. In another technique, the powdered embolic hydrogel may be deficient in any or all of these needed or desired substances to aid in overall stability (of powder or of substances in the solution), ability to reconstitute, etc. In this case, the reconstitution solution subsequently added to the powdered embolic hydrogel during the rehydration step may include an aqueous buffer that comprises these substances.

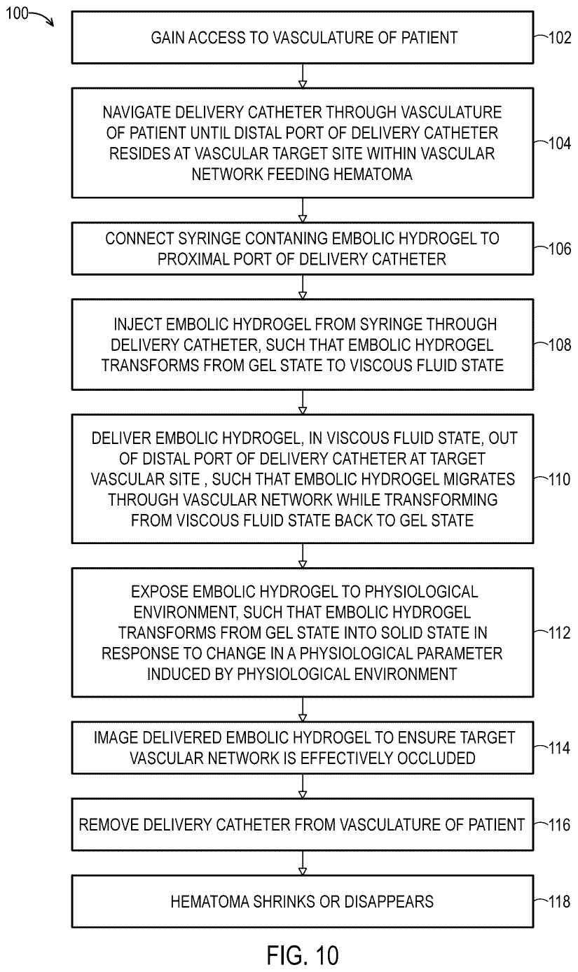

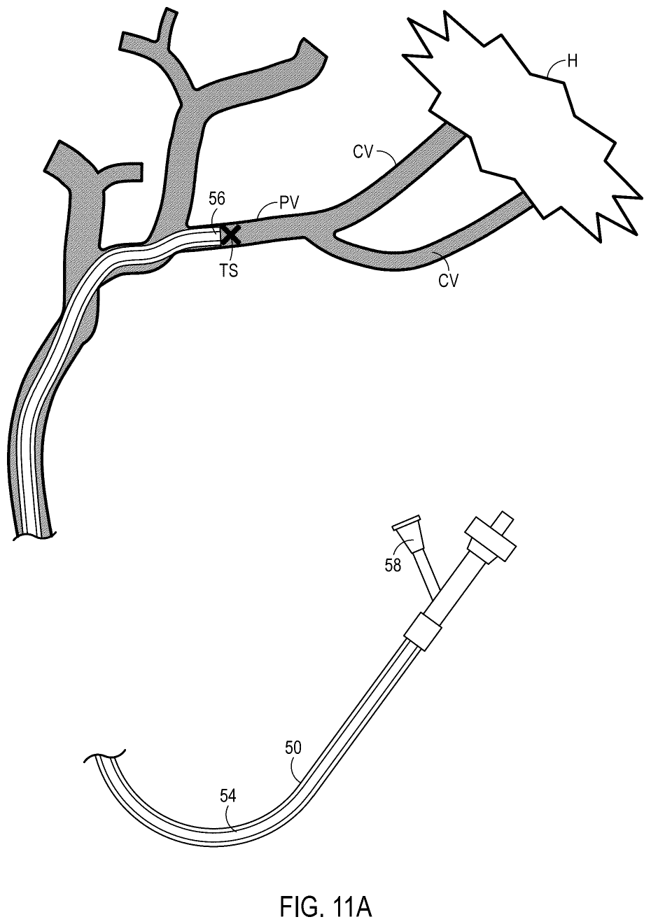

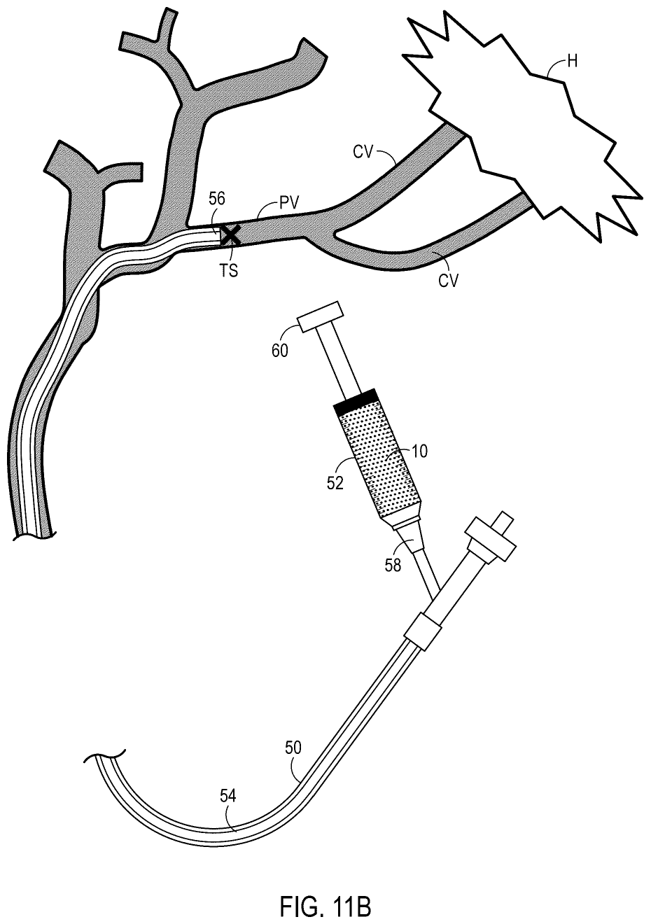

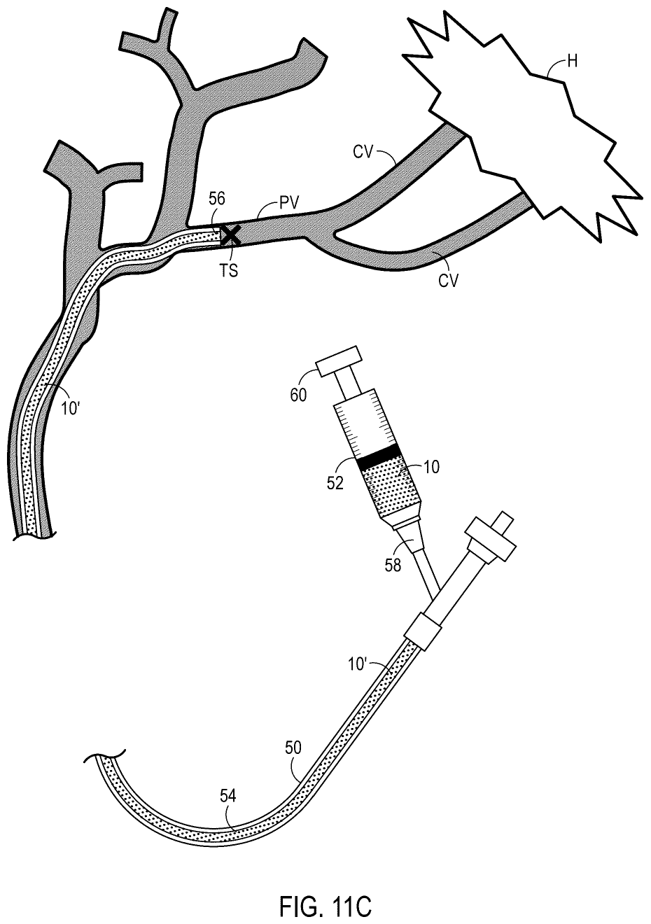

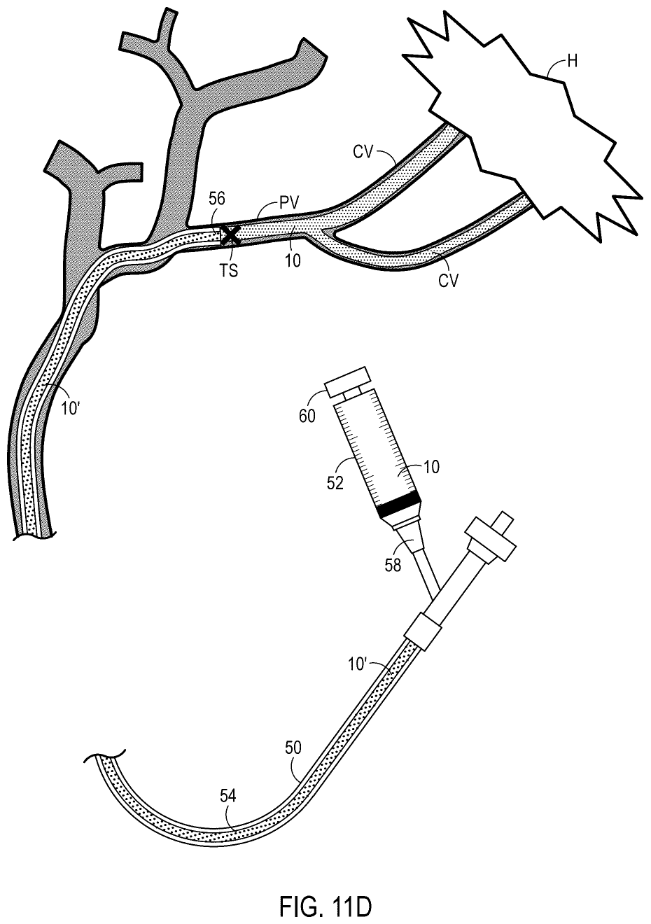

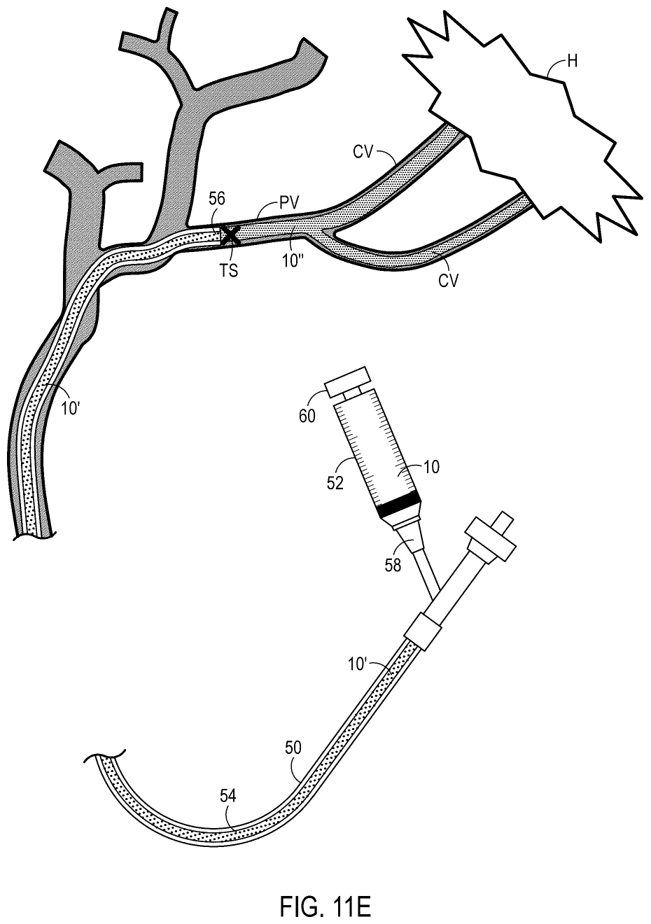

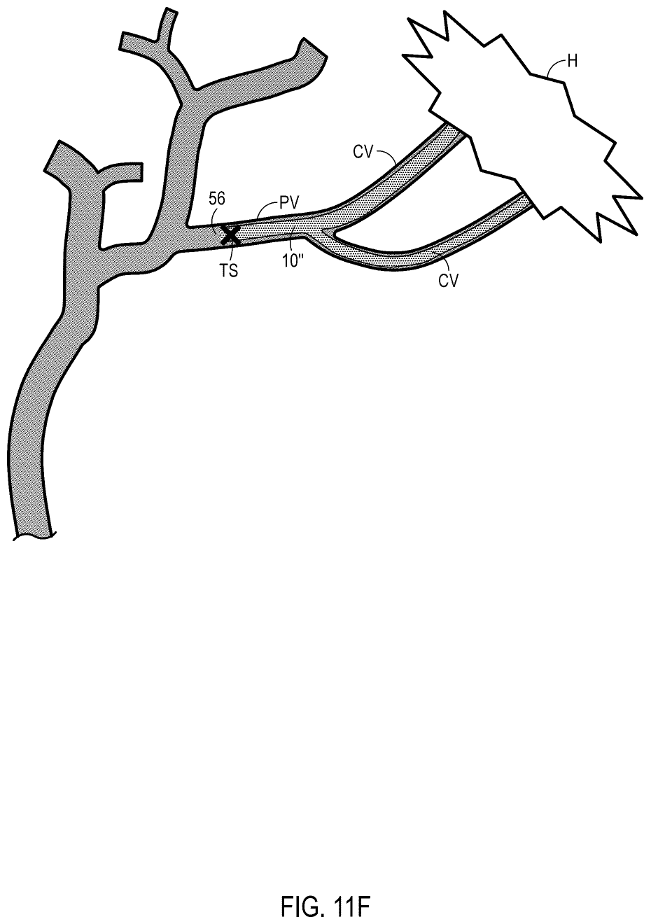

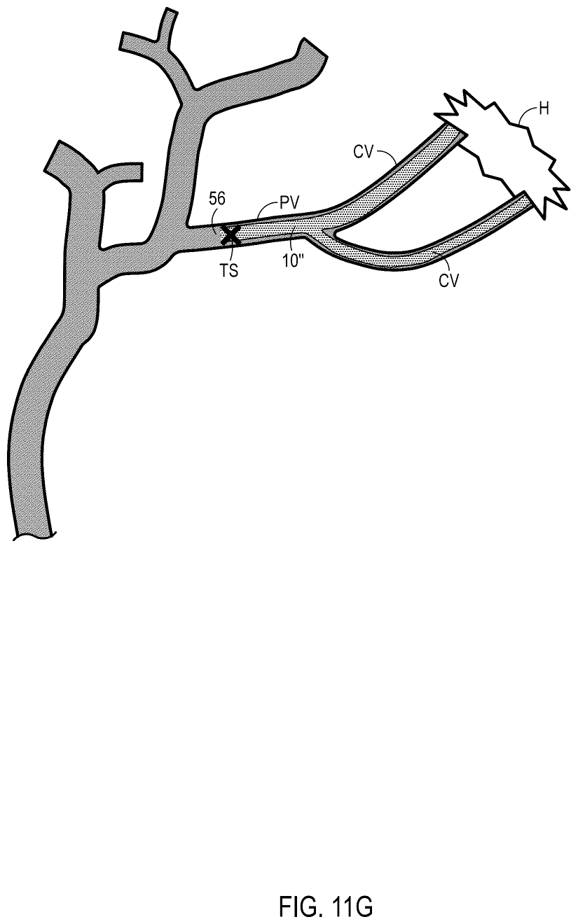

Having described various embodiments of the embolic hydrogel 10, one method 100 of delivering the embolic hydrogel 10 through a medical device to treat a patient having a medical anomaly will now be described with reference to FIGS. 10 and 11A-11G. In this method, anomaly is a hematoma (e.g., a chronic subdural hematoma resulting from leakage of blood from the middle meningeal artery (MMA)), although it should be appreciated that the hematoma may be located anywhere in the body of the patient. In this method, the embolic hydrogel 10 is endovascularly delivered at a target vascular site (e.g., in the MMA), in which case, the medical device through which the embolic hydrogel 10 is delivered is an intravascular delivery catheter 50 (e.g., in an embolization of the middle meningeal artery embolization (EMMA) procedure). The delivery catheter 50 is preferably a small diameter microcatheter (e.g., having a delivery lumen 54 with a diameter equal to or less than 0.025″, and preferably, equal to or less than 0.018″) capable of reaching target sites within the cerebral vasculature of the patient. The embolic hydrogel 10 may be conveniently supplied in a syringe 52 that is coupleable to the delivery catheter 50.

First, a clinician gains access to the patient's vasculature, typically through the patient's femoral in the groin, using an introducer kit and known access techniques (step 102). Alternative entry sites are sometimes chosen (e.g., in the arm or neck), which are in general well known by clinicians. The clinician then navigates the delivery catheter 50 through the vasculature of the patient until a distal port 56 of the delivery catheter 50 resides at an vascular target site TS within the target vascular network (in this case, the parent vessel PV and children vessels CV) feeding the hematoma H (step 104) (see FIG. 11A). Preferably, the vascular target site TS is distal to any non-targeted vessels that do not feed the hematoma H to minimize the occlusion of any blood flow to healthy anatomical structures.

The delivery catheter 50 may have an “over-the-wire” configuration, in which case, the entire length of the delivery catheter 50 may be advanced over a guidewire (not shown) into the patient. Alternatively, the delivery catheter 50 may have a “rapid-exchange” configuration, in which case, the guidewire extends through only a distal portion of the delivery catheter 50 from a guidewire port (not shown). In other alternative embodiments, the delivery catheter 50 may be introduced into the patient after a guidewire has been withdrawn leaving a sheath or access catheter distal portion at the vascular target site for the delivery catheter 50 to navigate through the vasculature of the patient within the sheath or access catheter. In the illustrated embodiment, the delivery catheter 50 is conventional in that it comprises only one delivery lumen.

Next, the syringe 52 containing the embolic hydrogel 10 is connected to a proximal port 58 of the delivery catheter 50 (step 106) (see FIG. 11B). Notably, the proximal port 58 is in fluid communication with the distal port 56 via the delivery lumen 54 of the delivery catheter 50. Next, the embolic hydrogel 10 is injected from the syringe 52 (via distal advancement of the plunger 60 of the syringe 52) and through the delivery catheter 50, such that the embolic hydrogel transitions from a gel state 10 to a viscous liquid state 10′ during injection through the delivery catheter 50 (step 108) (see FIG. 11C). That is, the shear forces exerted on the embolic hydrogel by the delivery lumen 54 of the delivery catheter 50 momentarily break the primary reversible crosslinks within the embolic hydrogel, thereby transitioning the embolic hydrogel from its gel state 10 to its viscous liquid state 10′ within the delivery lumen 54 of the delivery catheter 50. As a result, delivery of the embolic hydrogel through the relatively small diameter delivery catheter 50 is enabled in response to the application of a relatively small force on the embolic hydrogel via the plunger 60 of the syringe 52 (e.g., less than 50N).

Then, the embolic hydrogel, in its viscous liquid state 10′, is delivered out of the distal port 56 of the delivery catheter 50 into the patient at the vascular target site TS, such that the embolic hydrogel migrates through the target vascular network (and in particular, down the parent vessel PV into the children vessels CV just proximal to the hematoma H), while transitioning back from its viscous liquid state 10′ to its gel state 10 (step 110) (see FIG. 11D). That is, as the embolic hydrogel exits the distal port 56 of the delivery catheter 50, the shear forces previously exerted on the embolic hydrogel by the syringe 52 and delivery catheter 50 are removed, such that primary reversible crosslinks reform within the embolic hydrogel, thereby transitioning the embolic hydrogel from its viscous liquid state 10′ back to its gel state 10. Preferably, the embolic hydrogel transitions from its viscous liquid state 10′ back to its gel state 10 within fifteen seconds, and preferably within five seconds, as it exits the distal port 56 of the delivery catheter 50. It should be appreciated that, although the embolic hydrogel may not completely transition from its viscous liquid state 10′ back to its gel state 10 until a finite time (e.g., five seconds) has elapsed, the initiation of such transition almost immediately occurs (as illustrated in FIGS. 8C and 9C) as the embolic hydrogel exits the distal port 56 of the delivery catheter 50. Thus, the viscosity and storage modulus of the embolic hydrogel, once it exits the distal port 56 of the delivery catheter 50, appropriately changes in a continual manner, such that it becomes more difficult for the embolic hydrogel to reflux back towards the distal port 56 of the delivery catheter 50 over time. Thus, control of the embolic hydrogel, once it exits the distal port 56 of the delivery catheter 50, is maintained well before complete transition from its viscous liquid state 10′ back to its gel state 10. In its gel state 10, the embolic hydrogel then migrates with the flow of blood into the target vascular network leading to the hematoma H. Notably, because the embolic hydrogel is in its gel state 10 as it exits the distal port 56 of the delivery catheter 50, any danger that the embolic hydrogel proximally migrates (reflux) back into non-targeted vessels that feed healthy anatomical structures and/or entrapment of the delivery catheter 50 is minimized.

The delivered embolic hydrogel is then exposed to a physiological environment within the target vascular network, such that the delivered embolic hydrogel transitions from its gel state 10 into its solid state 10″ within the target vascular network leading to the hematoma H in response to a change in a physiological parameter (e.g., a change in pH, temperature, or ionic content) induced by the physiological environment, thereby occluding the target vascular network, and preventing further leakage of blood to the hematoma H (step 112) (see FIG. 11E). Preferably, there is a delayed reaction for transitioning the embolic hydrogel from its gel state 10 to its solid state 10″ when exposed to the physiological environment. For example, it is preferred that such transition completely occur more than ten seconds, and preferably more than one minute, after exposure to the physiological environment to provide the time necessary for the embolic hydrogel to distally migrate through the target vascular network with the blood flow.

Anytime during the medical procedure, the delivered embolic hydrogel, in its gel state 10 or its solid state 10″, may be imaged, e.g., via fluoroscopy to ensure that the target vascular network has been effectively occluded (step 114). In this case, the embolic hydrogel preferably contains a contrast agent (e.g., an iodine-based contrast agent or a metal-based contrast agent). Optionally, the embolic hydrogel contains a pharmacological agent and/or a radioisotope to provide localized therapy to the hematoma H or the tissue surrounding the hematoma H. Lastly, the delivery catheter 50 is removed from the vasculature of the patient (step 116) (see FIG. 11F). After a period of time, the hematoma H shrinks or even disappears due to the cessation of blood flow to the hematoma H (caused by the occlusion of the target vascular network) and the absorption of the hematoma H into the surrounding tissue of the patient (step 118) (see FIG. 11G).

Of course, methods similar to the method 100 described with respect to FIGS. 10 and 11A-11G may be employed to endovascularly occlude blood vessels to treat other medical anomalies, e.g., vascular defects, such as hemorrhage and diseased blood vessels, such as an arteriovenous malformation (AVM), an arteriovenous fistula, or an aneurysm, or non-vascular defects, such as tumors. Furthermore, although the method 100 is described for use with an intravascular delivery catheter 50 for endovascular injection of the embolic hydrogel 10 to treat medical anomalies, it should be appreciated that alternative methods may non-endovascularly inject the embolic hydrogel 10 into an interstitial space within the patient via a syringe, e.g., to treat an open wound.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An embolic hydrogel for use with a patient, comprising:
an aqueous buffer;
a first functionalized polymer having a polymer backbone chemically modified with a first functional group;
a second functionalized polymer having a polymer backbone chemically modified with a second functional group different from the first functional group, wherein the first functional group and the second functional group interact with each other to facilitate formation of primary reversible crosslinks between the respective polymer backbones of the first functionalized polymer and the second functionalized polymer, such that the embolic hydrogel transitions from a gel state to a viscous liquid state in response to a shear force applied to the embolic hydrogel, and transitions back from the viscous liquid state to the gel state in response to an absence of a shear force applied to the embolic hydrogel; and
at least one phase-transitioning polymer that creates secondary crosslinks therein in response to a physiological environment, such that the embolic hydrogel transitions from the gel state to a solid state.

2. The embolic hydrogel of claim 1, wherein the embolic hydrogel transitions from the viscous liquid state to the gel state within fifteen seconds in response to the absence of the shear forces after injection into the patient.

3. The embolic hydrogel of claim 2, wherein the embolic hydrogel transitions from the gel state to the solid state after ten seconds in response to the physiological environment is greater than ten seconds.

4. The embolic hydrogel of claim 1, wherein the embolic hydrogel, when in the viscous liquid state, is configured for being injected through a delivery catheter having an inner lumen equal to or less than 0.018″ in diameter at a force less than 30N.

5. The embolic hydrogel of claim 1, wherein one of the at least one phase-transitioning polymer comprises the polymer backbone of the first functionalized polymer.

6. The embolic hydrogel of claim 5, wherein the at least one phase-transitioning polymers comprises a single phase-transitioning polymer, and the single phase-transitioning polymer comprises the polymer backbone of only the first functionalized polymer.

7. The embolic hydrogel of claim 1, wherein one or more of the at least one phase-transitioning polymer is not chemically modified with either of the first functional group or the second functional group, such that the one or more of the at least one phase-transitioning polymer does not contribute to the formation of the primary reversible crosslinks on the polymer backbones of the first functionalized polymer and the second functionalized polymer.

8. The embolic hydrogel of claim 1, wherein the primary reversible crosslinks are covalent, and the secondary crosslinks are physical crosslinks.

9. The embolic hydrogel of claim 1, wherein the at least one phase-transitioning polymer creates the secondary crosslinks therein in response to change in one or more of a pH, temperature, and an ionic content induced by the physiological environment.

10. An embolic hydrogel for use with a patient, comprising:
an aqueous buffer;
at least one functionalized polymer respectively having at least one polymer backbone chemically modified with one or more functional groups, wherein the one or more functional groups facilitate formation of primary reversible crosslinks on the at least one polymer backbone, such that the embolic hydrogel transitions from a gel state to a viscous liquid state in response to a shear force applied to the embolic hydrogel, and transitions back from the viscous liquid state to the gel state in response to an absence of a shear force applied to the embolic hydrogel; and
at least one phase-transitioning polymer that creates secondary crosslinks therein in response to a physiological environment, such that the embolic hydrogel transitions from the gel state to a solid state, wherein one or more of the at least one phase-transitioning polymer does not contribute to the formation of the primary reversible crosslinks on the at least one polymer backbone.

11. The embolic hydrogel of claim 10, wherein none of at least one polymer backbone of the at least one functionalized polymer is a phase-transitioning polymer.

12. The embolic hydrogel of claim 10, wherein each of the one or more of the at least one phase-transitioning polymer comprises a polymer backbone that is not chemically modified with any of the one or more functional groups.

13. The embolic hydrogel of claim 10, wherein the polymer backbone of each of the one or more of the at least one phase-transitioning polymer is separate from the polymer backbones of the first functionalized polymer and the second functionalized polymer.

14. The embolic hydrogel of claim 10, wherein the embolic hydrogel transitions from the viscous liquid state to the gel state within fifteen seconds in response to the absence of the shear forces after injection into the patient.

15. The embolic hydrogel of claim 14, wherein the embolic hydrogel transitions from the gel state to the solid state after ten seconds in response to the physiological environment is greater than ten seconds.

16. The embolic hydrogel of claim 10, wherein the embolic hydrogel, when in the viscous liquid state, is configured for being injected through a delivery catheter having an inner lumen equal to or less than 0.018″ in diameter at a force less than 30N.

17. The embolic hydrogel of claim 10, wherein the one or more functional groups comprises at least two different functional groups that interact act with each other to form the primary reversible crosslinks on the at least one polymer backbone.

18. The embolic hydrogel of claim 10, wherein the at least one functionalized polymer comprises a single functionalized polymer, and the one or more functional groups comprises a metal coordinating ligand, the embolic hydrogel further comprising a solution of metal ions or metal nanoparticles, wherein the metal coordinating ligand and the metal ions or metal nanoparticles interact with each other to form the primary reversible crosslinks within the polymer backbone of the single functionalized polymer.

19. The embolic hydrogel of claim 10, wherein the primary reversible crosslinks are covalent, and the secondary crosslinks are physical crosslinks.

20. The embolic hydrogel of claim 10, wherein the at least one phase-transitioning polymer creates the secondary crosslinks therein in response to change in one or more of a pH, temperature, and an ionic content induced by the physiological environment.
